(19) United States
(12) Patent Application Publication
(10) Pub. No.: US 2026/0183550 A1
(43) Pub. Date: Jul. 2, 2026

(54) RESPIRATORY SINUS ARRHYTHMIA (RSA) PACING MODE ACTIVATION

(71) Applicant: Medtronic, Inc., Minneapolis, MN (US)

(72) Inventors: Michael D. Eggen, Chisago City, MN (US); Yong K. Cho, Excelsior, MN (US)

(21) Appl. No.: 19/125,025

(22) PCT Filed: Oct. 25, 2023
(86) PCT No.: PCT/US2023/077774

§ 371 Date: Apr. 28, 2025

Related U.S. Application Data
(60) Provisional application No. 63/381,426, filed on Oct. 28, 2022.

(51) Int. Cl. A61N 1/365; A61N 1/362; A61N 1/39

(52) U.S. Cl. CPC A61N 1/36585; A61N 1/3621; A61N 1/3987

(57) ABSTRACT

A device comprises therapy delivery circuitry configured to deliver cardiac pacing pulses to a heart of a patient via a plurality of electrodes, sensing circuitry configured to sense one or more parameters of the patient, and processing circuitry. The processing circuitry is configured to determine that one or more criteria for activation of a respiratory sinus arrhythmia (RSA) mode are satisfied based on the one or more parameters of the patient, and control the therapy delivery circuitry to deliver the cardiac pacing pulses according to the RSA mode based on the determination. Delivery of the cardiac pacing pulses according to the RSA mode includes increasing a rate of the cardiac pacing pulses during an inspiration phase of a respiration cycle of the patient, and decreasing a rate of the cardiac pacing pulses during an expiration phase of the respiration cycle of the patient.

This application claims priority from and the benefit of U.S. Provisional Patent Application Ser. No. 63/381,426, filed Oct. 28, 2022, the entire content of which is incorporated herein by reference.

### FIELD

The disclosure relates to medical devices, and, more particularly, to medical devices that deliver cardiac therapy.

### BACKGROUND

In healthy humans, heart rate naturally increases during inspiration and decreases during expiration. This phenomenon, known as respiratory sinus arrhythmia (RSA), supports ventilation/perfusion matching as blood enters the lungs, i.e., increases pulmonary blood flow when the lungs are inflated. RSA diminishes or disappears in heart failure (HF) patients. It has been proposed in literature and shown in HF animal models that restoring RSA increases cardiac output and helps to reverse remodel the heart.

### SUMMARY

In general, this disclosure describes techniques for delivering cardiac pacing to restore RSA, e.g., by increasing the cardiac pacing pulse rate during inspiration and decreasing the pulse rate during expiration. While cardiac pacing to restore RSA may provide therapeutic benefit, e.g., by increasing cardiac output and helping to reverse remodel the heart of heart failure patients, the RSA pacing may be unnecessary, ineffective, or counterproductive under certain conditions. The techniques of this disclosure may avoid delivering RSA pacing under such conditions by determining whether one or more criteria for activating an RSA mode of cardiac pacing are satisfied based on one or more sensed patient parameters. In this manner, the techniques described herein may advantageously improve the operation of a device that delivers cardiac pacing to restore RSA, e.g., to deliver such pacing when it will likely be effective and avoid delivery of counterproductive therapy, thereby benefitting the patient.

In one example, a device comprises therapy delivery circuitry configured to deliver cardiac pacing pulses to a heart of a patient via a plurality of electrodes, sensing circuitry configured to sense one or more parameters of the patient, and processing circuitry. The processing circuitry is configured to determine that one or more criteria for activation of a respiratory sinus arrhythmia (RSA) mode are satisfied based on the one or more parameters of the patient, and control the therapy delivery circuitry to deliver the cardiac pacing pulses according to the RSA mode based on the determination. Delivery of the cardiac pacing pulses according to the RSA mode includes increasing a rate of the cardiac pacing pulses during an inspiration phase of a respiration cycle of the patient, and decreasing a rate of the cardiac pacing pulses during an expiration phase of the respiration cycle of the patient.

In another example, a method comprises sensing one or more parameters of a patient, determining that one or more criteria for activation of a respiratory sinus arrhythmia (RSA) mode are satisfied based on the one or more parameters of the patient, and delivering cardiac pacing pulses according to the RSA mode based on the determination. Delivery of the cardiac pacing pulses according to the RSA mode includes increasing a rate of the cardiac pacing pulses during an inspiration phase of a respiration cycle of the patient, and decreasing a rate of the cardiac pacing pulses during an expiration phase of the respiration cycle of the patient.

In other examples, a non-transitory computer-readable storage medium comprising program instructions that, when executed by processing circuitry of a device, cause the device to perform the methods described herein.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a conceptual drawing illustrating an example system configured to deliver cardiac pacing according to a respiratory sinus arrhythmia (RSA) pacing mode, the system including an implantable medical device (IMD) coupled to implantable medical leads.

FIG. 2 is a conceptual drawing illustrating the example IMD and leads of FIG. 1 in conjunction with a heart.

FIG. 3 is a functional block diagram illustrating an example configuration of the IMD of FIG. 1.

FIG. 4 is a flow diagram illustrating an example operation of a device to determine whether to activate an RSA pacing mode.

FIG. 5 is a flow diagram illustrating an example operation of a device to determine whether an RSA pacing mode activation criterion is satisfied.

FIG. 6 is a flow diagram illustrating an example operation of a device to configure delivery of RSA pacing.

### DETAILED DESCRIPTION

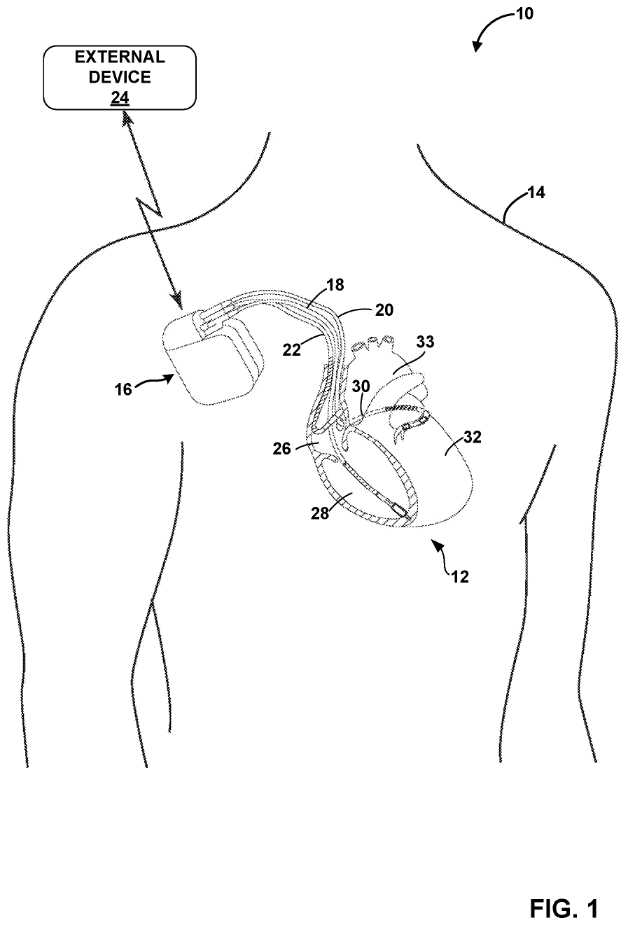

FIG. 1 is a conceptual drawing illustrating an example system 10 configured to deliver cardiac pacing according to a respiratory sinus arrhythmia (RSA) pacing mode in order to restore RSA in a patient 14. In the example of FIG. 1, system 10 includes IMD 16, which is coupled to leads 18, 20, and 22, and an external device 24. IMD 16 may be, for example, an implantable pacemaker, cardioverter, and/or defibrillator that provides electrical signals to heart 12 via electrodes coupled to one or more of leads 18, 20, and 22. Patient 14 is ordinarily, but not necessarily a human patient.

In the example of FIG. 1, leads 18, 20, 22 extend into the heart 12 of patient 14 to sense electrical activity of heart 12, e.g., one or more cardiac electrogram signals, and/or deliver electrical stimulation to heart 12. Leads 18, 20, and 22 may also be used to detect impedance indicative of fluid volume in patient 14 and respiration of patient 14. In addition to impedance, a respiration signal may also be present as a component of a cardiac electrogram signal.

In the example shown in FIG. 1, right ventricular (RV) lead 18 extends through one or more veins (not shown), the superior vena cava (not shown), and right atrium 26, and into right ventricle 28. Left ventricular (LV) coronary sinus lead 20 extends through one or more veins, the vena cava, right atrium 26, and into the coronary sinus 30 to a region adjacent to the free wall of left ventricle 32 of heart 12. Right atrial (RA) lead 22 extends through one or more veins and the vena cava, and into the right atrium 26 of heart 12.

The illustrated number and positions of leads 18, 20, and 22 are examples. In other examples, IMD 10 may be coupled to one, two, or more than three leads that extend to a variety of positions. In some examples, system 10 may additionally or alternatively include one or more leads or lead segments (not shown in FIG. 1) that deploy one or more electrodes within the vena cava, or other veins. Furthermore, in some examples, system 10 may additionally or alternatively include extravascular leads with electrodes implanted outside of heart 12, instead of or in addition to transvenous, intracardiac leads 18, 20 and 22. Such leads may be used for one or more of cardiac sensing, pacing, or cardioversion/defibrillation. Additionally, in some examples, system 10 may include one or more leadless cardiac pacing devices, such as the Micra™ pacemakers commercially available from Medtronic, Inc., instead of or in addition to IMD 16. One or more leadless pacemakers may be configured to deliver cardiac pacing according to an RSA mode in the manner described herein with respect to IMD 16. Furthermore, an external medical device may be configured to deliver cardiac pacing according to an RSA mode in the manner described herein with respect to IMD 16. In some examples, a system may additionally or alternatively include one or more implantable or external monitoring devices that monitor patient parameters but do not provide therapy, such as a Reveal LINQ™ insertable cardiac monitor, commercially available from Medtronic, Inc.

IMD 16 may sense electrical signals attendant to the depolarization and repolarization of heart 12 via electrodes (not shown in FIG. 1) coupled to at least one of the leads 18, 20, 22. In some examples, IMD 16 provides pacing pulses to heart 12 based on the electrical signals sensed within heart 12. The configurations of electrodes used by IMD 16 for sensing and pacing may be unipolar or bipolar. In some examples, IMD 16 may deliver cardiac pacing to provide cardiac resynchronization therapy (CRT). In some examples, IMD 16 may additionally or alternatively be configured to provide conduction system pacing, which may provide a more physiologic activation of heart 12 than conventional pacing. In such examples, leads 18, 20, 22 may be configured/positioned such that their electrode(s) access (are capable of stimulating) the heart's conduction system, e.g., the His bundle, left bundle branch, or right bundle branch.

IMD 16 may detect arrhythmia of heart 12, such as tachycardia or fibrillation of the atria 26 and 36 and/or ventricles 28 and 32, and may also provide defibrillation therapy and/or cardioversion therapy via electrodes located on at least one of the leads 18, 20, 22. In some examples, IMD 16 may be programmed to deliver a progression of therapies, e.g., pulses with increasing energy levels, until a fibrillation of heart 12 is stopped. IMD 16 may detect fibrillation employing one or more fibrillation detection techniques known in the art.

IMD 16 may utilize two of any electrodes carried on leads 18, 20, 22 to generate electrograms of cardiac activity. In some examples, IMD 16 may also use a housing electrode of IMD 16 (not shown) to generate electrograms and monitor cardiac activity. Although these electrograms may be used to monitor heart 12 for potential arrhythmias and other disorders for therapy, the electrograms may also be used to monitor the condition of heart 12. For example, IMD 16 may monitor heart rate, heart rate variability, indicators of blood flow, or other indicators of the ability of heart 12 to pump blood or the progression of heart failure.

In some examples, IMD 16 may also use any two electrodes of leads 18, 20, and 22 or the housing electrode to sense an impedance of patient 14. As the tissues within the thoracic cavity of patient 14 increase in fluid content, the impedance between two electrodes may also change. IMD 16 may use this impedance to create a fluid index. As the fluid index increases, more fluid may be more likely to be retained within patient 14 and heart 12 may be stressed to keep up with moving the greater amount of fluid. An example system for measuring thoracic impedance and determining a fluid index is described in U.S. Patent Publication No. 2010/0030292 by Sarkar et al., entitled, “DETECTING WORSENING HEART FAILURE BASED ON IMPEDANCE MEASUREMENTS,” which published on Feb. 4, 2010 and is incorporated herein by reference in its entirety.

IMD 16 may communicate with external device 24. In some examples, external device 24 comprises a handheld computing device, computer workstation, or networked computing device. External device 24 may be configured to retrieve data from IMD 16, e.g., for presentation to a clinician or other user, such as sensed parameter data of patient 14 and data regarding the operation of IMD 16. In some examples, external device 24 may provide the retrieved data to a cloud computing system, such as the Carelink™ system available from Medtronic, Inc., which may analyze the data and provide reports of the analysis and/or the data to clinicians or other users. In some examples, a clinician or other user may also interact with programmer 24 to program IMD 16, e.g., select values for operational parameters of IMD 16. Although the user is typically a clinician, the user may be patient 14 in some examples.

In some examples, IMD 16, external device 24, or a cloud computing system may determine heart failure metrics based on patient parameter data collected by IMD 16, and determine a heart failure risk level based on the heart failure risk metrics. For example, the risk level may be determined based on a predetermined number of metrics exceeding their representative thresholds or a weighted score for each of the patient metrics for exceeding one or more thresholds. Additionally, or alternatively, the risk level may be determined by a Bayesian Belief Network, or other probability technique, using the values or stratified states of each automatically detected patient metric. For example, a Bayesian Belief Network may be applied to the values of the patient metrics to determine the risk level, e.g., the probability, that patient 14 will be admitted to the hospital for heart failure.

IMD 16 may determine each of the heart failure metrics and store them within the IMD for later transmission. For example, the patient metrics may include two or more of a thoracic fluid index, an atrial fibrillation duration, a ventricular contraction rate during atrial fibrillation, a patient activity, a nighttime heart rate, a heart rate variability, a CRT percentage (e.g., the percentage of cardiac cycles for which CRT pacing was provided), or the occurrence of or number of therapeutic electrical shocks. One method for determining heart failure risk status is described in U.S. Publication No. 2012/0253207 A1, entitled “Heart Failure Monitoring,” by Sarkar et al., which is incorporated herein by reference in its entirety.

IMD 16 and programmer 24 may communicate via wireless communication using any techniques known in the art. Examples of communication techniques may include, for example, radiofrequency (RF) telemetry or communication according to a Bluetooth® protocol, but other communication techniques such as magnetic coupling are also contemplated.

IMD 16 is an example of a device configured to deliver cardiac pacing pulses to a heart of a patient via a plurality of electrodes, sense one or more parameters of the patient, determine that one or more criteria for activation of an RSA mode are satisfied based on the one or more parameters of the patient, and deliver the cardiac pacing pulses according to the RSA mode based on the determination.

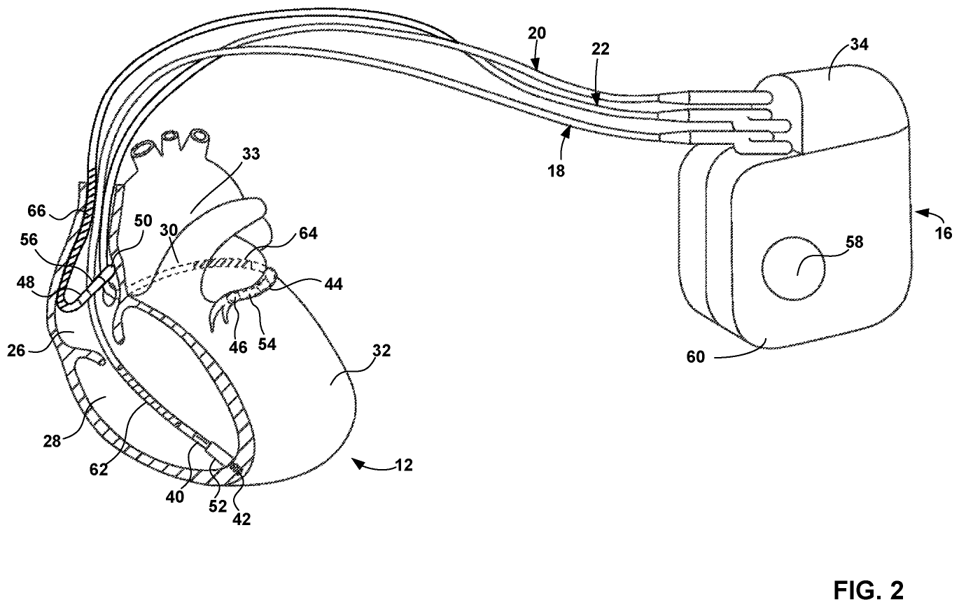

FIG. 2 is a conceptual drawing illustrating IMD 16 and leads 18, 20, and 22 of system 10 in greater detail. As shown in FIG. 2, IMD 16 is coupled to leads 18, 20, and 22. Leads 18, 20, 22 may be electrically coupled to therapy delivery circuitry and sensing circuitry of IMD 16 via connector block 34. In some examples, proximal ends of leads 18, 20, 22 may include electrical contacts that electrically couple to respective electrical contacts within connector block 34 of IMD 16. In addition, in some examples, leads 18, 20, 22 may be mechanically coupled to connector block 34 with the aid of set screws, connection pins, snap connectors, or another suitable mechanical coupling mechanism.

Each of the leads 18, 20, 22 includes an elongated insulative lead body, which may carry a number of concentric coiled conductors separated from one another by tubular insulative sheaths. Bipolar electrodes 40 and 42 are located adjacent to a distal end of lead 18 in right ventricle 28. In addition, bipolar electrodes 44 and 46 are located adjacent to a distal end of lead 20 in coronary sinus 30 and bipolar electrodes 48 and 50 are located adjacent to a distal end of lead 22 in right atrium 26. In the illustrated example, there are no electrodes located in left atrium 33. However, other examples may include electrodes in left atrium 33. Furthermore, in examples in which IMD 16 is configured to deliver conduction system pacing, lead 18 may configured/positioned differently than illustrated in FIG. 2 so that electrode 42 may stimulate the conduction system, e.g., His bundle, left bundle branch, or right bundle branch. For example, electrode 42 may be positioned on or in the ventricular septum.

Electrodes 40, 44, and 48 may take the form of ring electrodes, and electrodes 42, 46 and 50 may take the form of fixed or extendable helix tip electrodes mounted to insulative electrode heads 52, 54 and 56, respectively. In other examples, one or more of electrodes 42, 46 and 50 may take the form of small circular electrodes at the tip of a tined lead or other fixation element. Leads 18, 20, 22 also include elongated electrodes 62, 64, 66, respectively, which may take the form of a coil. Each of the electrodes 40, 42, 44, 46, 48, 50, 62, 64 and 66 may be electrically coupled to a respective one of the coiled conductors within the lead body of its associated lead 18, 20, 22, and thereby coupled to respective ones of the electrical contacts on the proximal end of leads 18, 20 and 22.

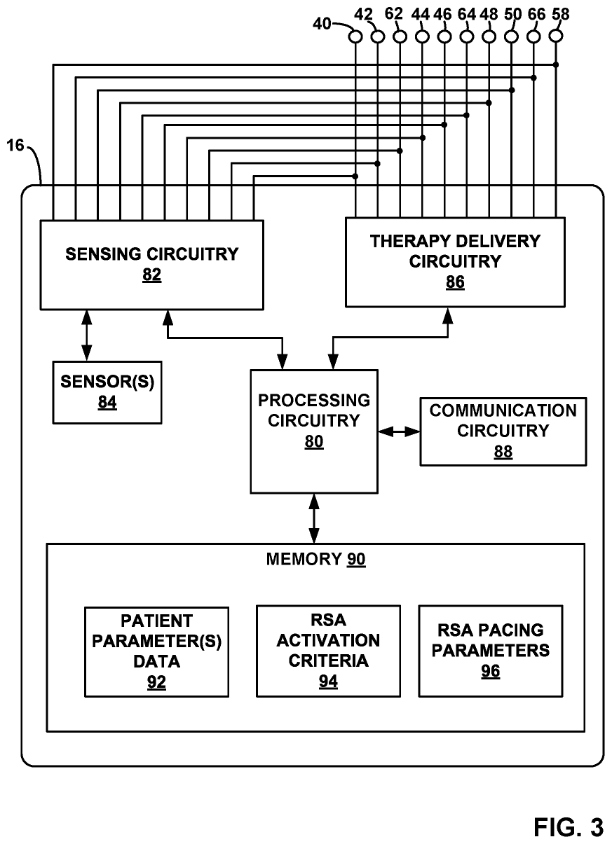

In some examples, as illustrated in FIG. 2, IMD 16 includes one or more housing electrodes, such as housing electrode 58, which may be formed integrally with an outer surface of hermetically-sealed housing 60 of IMD 16, or otherwise coupled to housing 60. In some examples, housing electrode 58 is defined by an uninsulated portion of an outward facing portion of housing 60 of IMD 16. Other division between insulated and uninsulated portions of housing 60 may be employed to define two or more housing electrodes. In some examples, housing electrode 58 comprises substantially all of housing 60. As described in further detail with reference to FIG. 3, housing 60 may enclose therapy delivery circuitry configured to generate therapeutic signals, such as cardiac pacing pulses and defibrillation shocks, as well as sensing circuitry for sensing the rhythm of heart 12 and other patient parameters.

IMD 16 may sense electrical signals attendant to the depolarization and repolarization of heart 12 via electrodes 40, 42, 44, 46, 48, 50, 62, 64 and 66. The electrical signals are conducted to IMD 16 from the electrodes via the respective leads 18, 20, 22. IMD 16 may sense such electrical signals via any bipolar combination of electrodes 40, 42, 44, 46, 48, 50, 62, 64 and 66. Furthermore, any of the electrodes 40, 42, 44, 46, 48, 50, 62, 64 and 66 may be used for unipolar sensing in combination with housing electrode 58. The combination of electrodes used for sensing may be referred to as a sensing configuration or electrode vector.

In some examples, IMD 16 delivers pacing pulses via bipolar combinations of electrodes 40, 42, 44, 46, 48 and 50 to produce depolarization of cardiac tissue of heart 12. In some examples, IMD 16 delivers pacing pulses via any of electrodes 40, 42, 44, 46, 48 and 50 in combination with housing electrode 58 in a unipolar configuration. Furthermore, IMD 16 may deliver defibrillation pulses to heart 12 via any combination of elongated electrodes 62, 64, 66, and housing electrode 58. Electrodes 58, 62, 64, 66 may also be used to deliver cardioversion pulses to heart 12. Electrodes 62, 64, 66 may be fabricated from any suitable electrically conductive material, such as, but not limited to, platinum, platinum alloy or other materials known to be usable in implantable defibrillation electrodes. The combination of electrodes used for delivery of therapy or sensing, their associated conductors and connectors, and any tissue or fluid between the electrodes, may define an electrical path.

In addition to electrograms of cardiac signals, any of electrodes 40, 42, 44, 46, 48, 50, 62, 64, 66, and 58 may be used to sense non-cardiac signals. For example, two or more electrodes may be used to measure an impedance, e.g., within the thoracic cavity of patient 14. This impedance may be used to generate a fluid index patient metric that indicates the amount of fluid building up within patient 14. Since a greater amount of fluid may indicate increased pumping loads on heart 12, the fluid index may be used as an indicator of heart failure risk. IMD 16 may periodically measure the intrathoracic impedance to identify a trend in the fluid index over days, weeks, months, and even years of patient monitoring.

In some examples, the two electrodes used to measure the intrathoracic impedance may be located at two different positions within the chest of patient 14. For example, coil electrode 62 and housing electrode 58 may be used as the sensing vector for intrathoracic impedance because electrode 62 is located within RV 28 and housing electrode 58 is located at the IMD 16 implant site generally in the upper chest region. However, other electrodes spanning multiple organs or tissues of patient 14 may also be used, e.g., an additional implanted electrode used only for measuring thoracic impedance.

FIG. 3 is a functional block diagram illustrating an example configuration of IMD 16. In the illustrated example, IMD 16 includes processing circuitry 80, sensing circuitry 82, one or more sensors 84, therapy delivery circuitry 86, communication circuitry 88, and memory 90. Memory 90 includes computer-readable instructions that, when executed by processing circuitry 80, cause IMD 16 and processing circuitry 80 to perform various functions attributed to IMD 16 and processing circuitry 80 herein. Memory 90 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital or analog media.

Processing circuitry 80 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 80 may include multiple components, such as any combination of one or more microprocessors, one or more controllers, one or more DSPs, one or more ASICs, or one or more FPGAs, as well as other discrete or integrated logic circuitry. The functions attributed to processing circuitry 80 herein may be embodied as software, firmware, hardware or any combination thereof, e.g., may be embodied as software or firmware executed on processing circuitry.

Processing circuitry 80 controls therapy delivery circuitry 86 to deliver therapy to heart 12 according to a therapy parameters and programs which may be stored in memory 90. An example of therapy parameters stored in memory 90 are RSA pacing parameters 96 for delivery of cardiac pacing according to an RSA pacing mode as discussed herein. Therapy delivery circuitry 86 is electrically coupled to electrodes 40, 42, 44, 46, 48, 50, 58, 62, 64, and 66, e.g., via conductors of the respective lead 18, 20, 22, or, in the case of housing electrode 58, via an electrical conductor disposed within housing 60 of IMD 16. In the illustrated example, therapy delivery circuitry 86 is configured to generate and deliver electrical therapy to heart 12. For example, therapy delivery circuitry 86 may deliver defibrillation shocks to heart 12 via at least two electrodes 58, 62, 64, 66. Therapy delivery circuitry 86 may deliver pacing pulses via ring electrodes 40, 44, 48 coupled to leads 18, 20, and 22, respectively, and/or helical electrodes 42, 46, and 50 of leads 18, 20, and 22, respectively. In some examples, therapy delivery circuitry 86 delivers pacing, cardioversion, or defibrillation stimulation in the form of electrical pulses. In other examples, therapy delivery circuitry 86 may deliver one or more of these types of stimulation in the form of other signals, such as sine waves, square waves, or other substantially continuous time signals.

Therapy delivery circuitry 86 includes circuitry, such as charge pumps, capacitors, current mirrors, or other signal generation circuitry for generating a pulse or other signal. Therapy delivery circuitry 86 may include a switch module and processor 80 may use the switch module to select, e.g., via a data/address bus, which of the available electrodes are used to deliver antitachyarrhythmia shocks or pacing pulses. The switch module may include a switch array, switch matrix, multiplexer, or any other type of switching device suitable to selectively couple stimulation energy to selected electrodes.

Sensing circuitry 82 monitors signals from at least one of electrodes 40, 42, 44, 46, 48, 50, 58, 62, 64 or 66 in order to monitor electrical activity of heart 12, impedance, respiration of patient 14, or other patient parameters, values of which may be stored as patient parameter data 92 in memory 90. Sensing may be done to detect intrinsic cardiac depolarizations, determine heart rates or heart rate variability, or to detect arrhythmias or other electrical signals. Sensing circuitry 82 may include one or more filters, amplifiers, analog-to-digital converters, or other sensing circuitry.

Sensing circuitry 82 may also include a switch module to select which of the available electrodes are used to sense the heart activity, depending upon which electrode combination, or electrode vector, is used in the current sensing configuration. In some examples, processing circuitry 80 may select the electrodes that function as sense electrodes, i.e., select the sensing configuration, via the switch module within sensing circuitry 82. Sensing circuitry 82 may include one or more detection channels, each of which may be coupled to a selected electrode configuration for detection of cardiac signals via that electrode configuration. Some detection channels may be configured to detect cardiac events, such as P- or R-waves, and provide indications of the occurrences of such events to processing circuitry 80.

One or more sensor(s) 84 may include, as examples, one or more accelerometers, microphones, temperature sensors, or optical sensors that are configured to provide signals or data representing one or more patient parameters to processing circuitry 80 via sensing circuitry 82. In some examples, based on a signal from one or more accelerometers, processing circuitry 80 may determine postures and/or activity levels of patient 14. In some examples, a signal from an optical sensor may include a respiration signal, and processing circuitry 80 may determine respiration metrics based on such a signal instead of or in addition to an impedance signal or a respiration component of a cardiac electrogram signal from sensing circuitry 82. In some examples, processing circuitry 80 may receive respiration signals or metrics from another implantable or external device, such as an implantable cardiac monitor, via communication circuitry 88. Example respiration metrics include respiration rate, identifications of respiration cycles including inspiration and expiration phases of respiration cycles, respiration effort such as inspiration effort and expiration effort, and tidal volume.

To determine respiration metrics, processing circuitry 80 may detect peaks and troughs in a respiration signal, e.g., by identifying zero slope points (zero crossings in a derivative or differential of the signal), identifying maximal or minimal values of the signal, or using any other peak/trough detection techniques. Processing circuitry 80 may determine an expiration phase as an interval or window from an identified peak to a subsequent trough, and an inspiration phase as an interval or window from an identified trough to a subsequent peak. Processing circuitry 80 may determine respiration effort based on one or more of a peak-to-trough amplitude or a slope of the signal within the inspiration phase. Processing circuitry 80 may determine tidal volume based on an area under the curve during the respiration cycle. In some examples, processing circuitry 80 may determine tidal volume based on a peak-to-trough amplitude, which may vary with tidal volume.

Processing circuitry 80 may implement programmable counters that control the basic time intervals associated with DDD, VVI, DVI, VDD, AAI, DDI, DDDR, VVIR, DVIR, VDDR, AAIR, DDIR, CRT, and other modes of pacing. Intervals defined by processing circuitry 80 may include atrial and ventricular pacing escape intervals, A-V intervals, V-V intervals, and refractory periods during which sensed P-waves and R-waves are ineffective to restart timing of the intervals. The durations of these intervals may be determined by processing circuitry 80 in response to stored data in memory 90.

In some examples, processing circuitry 80 may modify escape intervals based on a rate responsive pacing mode. Processing circuitry 80 may determine a sensor indicated pacing rate based on sensed parameters of patient 14, such as one or more of activity level or respiration rate, and thereby modify the escape interval and pacing rate to provide cardiac pacing that supports the activity of patient 14. In some examples, processing circuitry 80 may control IMD 16 to provide CRT by controlling delivery of pacing pulses to one or both of RV 28 and LV 32 based on atrioventricular timing and interventricular timing specified by one or more A-V intervals and V-V intervals.

Interval counters implemented by processing circuitry 80 may be reset upon sensing of R-waves and P-waves with detection channels of sensing circuitry 82. In examples in which IMD 16 provides pacing, therapy delivery circuitry 86 may include pacer output circuits that are coupled, e.g., selectively by a switching module, to any combination of electrodes 40, 42, 44, 46, 48, 50, 58, 62, or 66 appropriate for delivery of a bipolar or unipolar pacing pulse to one of the chambers of heart 12. In such examples, processing circuitry 80 may reset the interval counters upon the generation of pacing pulses by therapy delivery circuitry 86, and thereby control the basic timing of cardiac pacing functions, including anti-tachyarrhythmia pacing.

The value of the count present in the interval counters when reset by sensed R-waves and P-waves may be used by processing circuitry 80 to measure the durations of R-R intervals, P-P intervals, P-R intervals and R-P intervals, which are measurements that may be stored in memory 90. Processing circuitry 80 may use the count in the interval counters to detect a tachyarrhythmia event, such as atrial fibrillation (AF), atrial tachycardia (AT), ventricular fibrillation (VF), or ventricular tachycardia (VT). These intervals may also be used to detect the overall heart rate, ventricular contraction rate, and heart rate variability. A portion of memory 90 may be configured as a plurality of recirculating buffers, capable of holding series of measured intervals, which may be analyzed by processing circuitry 80 in response to the occurrence of a pace or sense interrupt to determine whether the patient's heart 12 is presently exhibiting atrial or ventricular tachyarrhythmia.

In some examples, processing circuitry 80 may determine that tachyarrhythmia has occurred by identification of shortened R-R (or P-P) interval lengths. Generally, processing circuitry 80 detects tachycardia when the interval length falls below 220 milliseconds (ms) and fibrillation when the interval length falls below 180 ms. These interval lengths are merely examples, and a user may define the interval lengths as desired, which may then be stored within memory 90. This interval length may need to be detected for a certain number of consecutive cycles, for a certain percentage of cycles within a running window, or a running average for a certain number of cardiac cycles, as examples.

In the event that processing circuitry 80 detects an atrial or ventricular tachyarrhythmia based on signals from sensing circuitry 82, and an anti-tachyarrhythmia pacing regimen is desired, timing intervals for controlling the generation of anti-tachyarrhythmia pacing therapies by therapy delivery circuitry 86 may be loaded by processing circuitry 80 to control the operation of the escape interval counters therein and to define refractory periods during which detection of R-waves and P-waves is ineffective to restart the escape interval counters for the an anti-tachyarrhythmia pacing. In the event that processing circuitry 80 detects an atrial or ventricular tachyarrhythmia based on signals from sensing circuitry 82, and a cardioversion or defibrillation shock is desired, processing circuitry 80 may control the amplitude, form and timing of the shock delivered by therapy delivery circuitry 86.

Memory 90 may be configured to store a variety of operational parameters, therapy parameters, sensed and detected data, and any other information related to the therapy and treatment of patient 14. In the example of FIG. 3, memory 82 includes patient parameter data 92, RSA activation criteria 94, and RSA pacing parameters. Patient parameter data 92 may store all of the data generated from the sensing and detecting of patient parameters described herein, such as activity, posture, heart rates, respiration metrics, fluid index, an atrial tachycardia or fibrillation burden, a ventricular contraction rate during atrial fibrillation, a nighttime heart rate, a difference between night and day heart rate, a heart rate variability, a cardiac resynchronization therapy percentage, a bradyarrhythmia pacing therapy percentage (in a ventricle and/or atrium), and number or frequency of electrical shock events, blood pressure, right ventricular pressure, pulmonary artery pressure, patient temperature, or biomarkers such as a brain natriuretic peptide (BNP), troponin, or related surrogates. In some examples, processing circuitry 80 may determine heart failure metrics based on sensed parameter data 92 and determine a heart failure risk level based on the heart failure metrics.

RSA activation criteria 94 includes one or more criteria that processing circuitry 80 may apply to patient parameter data 92 to determine whether to activate an RSA pacing mode. Processing circuitry 80 may activate the RSA pacing mode if patient parameter data 92 satisfies RSA activation criteria 94. RSA activation criteria 94 may be fixed, programmable by a user, or variable based on conditions determined by processing circuitry 80. To activate the RSA pacing mode, processing circuitry 80 control therapy delivery circuitry 86 to deliver pacing pulses, according to RSA pacing parameters 96, with increasing rates during an inspiration phase of a respiratory cycle, and decreasing rates during an expiration phase of the cardiac cycle, as described herein. The increasing and decreasing of pacing rates may be sequential, on a beat-to-beat or other basis.

Communication circuitry 88 includes any suitable hardware, firmware, software or any combination thereof for communicating with another device, such as external device 24 (FIG. 1). Under the control of processing circuitry 80, communication circuitry 88 may communicate with external device 24 with the aid of an antenna, which may be internal and/or external.

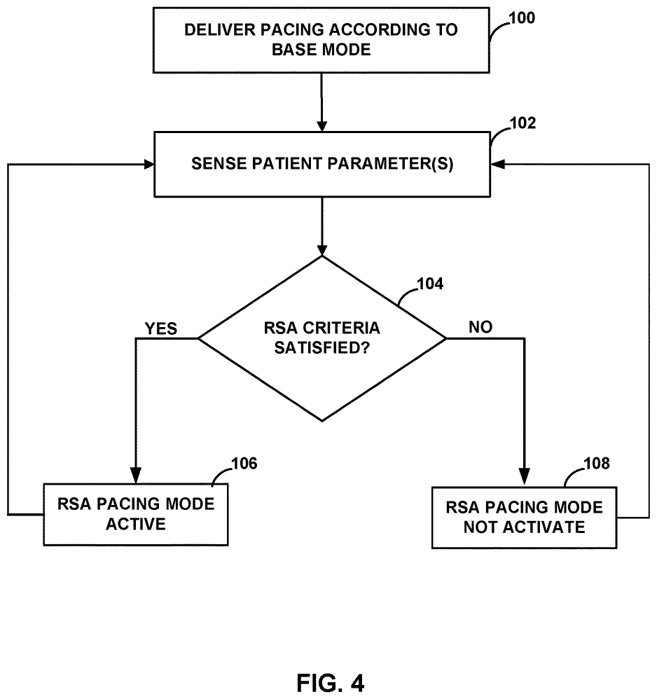

FIG. 4 is a flow diagram illustrating an example operation of a device to determine whether to activate an RSA pacing mode. Although described in the context of IMD 16, the example operation of FIG. 4 may be additionally or alternatively be performed by other devices, as described herein.

According to the example of FIG. 4, processing circuitry 80 of IMD 16 controls therapy delivery circuitry 86 to deliver cardiac pacing according to a base mode, such as a demand mode, rate responsive mode, CRT mode, or conduction system pacing mode (100). Sensing circuitry 82 and/or sensor(s) 84 also sense one or more patient parameters (102), based on which processing circuitry 80 may store patient parameter data 92 in memory 90 for analysis. Example patient parameter data 92 includes one or more of intrinsic or paced heart rates, postures, activity levels, respiration rates, intrinsic levels of RSA, heart failure metrics or heart failure risk scores, or percentages or other amounts of time that patient 14 has received cardiac pacing.

Processing circuitry 80 determines whether one or more RSA activation criteria 94 are satisfied based on patient parameter data 92 (104). Based on determining that RSA activation criteria 94 are satisfied (YES of 104), processing circuitry 80 activates an RSA pacing mode using RSA pacing parameters 96, or continues according to the RSA pacing mode if already activated (106). Based on determining that RSA activation criteria 94 are not satisfied (NO of 104), processing circuitry 80 deactivates the RSA pacing mode using RSA pacing parameters 96, or does not activate the RSA pacing mode if it is not currently active (108). Processing circuitry 80 may sense patient parameters (102) and determine whether RSA criteria 94 are satisfied (104) on the same or different basis, which may be periodic and/or in response to a trigger.

In some examples, RSA activation criteria 94 include a threshold heart rate as a criterion that is satisfied if intrinsic or sensor indicated heart rate is below the threshold heart rate. RSA pacing may not be effective if an intrinsic or sensor indicated heart rate exceeds a threshold heart rate. Increasing heart rate during inspiration according to an RSA pacing mode while the heart rate is already relatively high may cluster additional heart beats closer together in a counterproductive manner that may reduce cardiac output, because at higher rates there may be less time for diastolic filling. The heart rate compared to the threshold heart rate may be a single heart rate of a current cardiac cycle, or an average or other statistical representation of a plurality of heart rates.

As another example, RSA activation criteria 94 may include a threshold respiration rate as a criterion that is satisfied if a respiration rate determined by processing circuitry 80 from a sensed respiration signal is below the threshold respiration rate. A relatively high respiration rate, e.g., due to exercise or sleep apnea, may make timing increasing and decreasing pulse rates during inspiration and expiration phases of respiration cycles for RSA pacing difficult. The respiration rate compared to the threshold respiration rate may be a single respiration rate of a respiration cycle, or an average or other statistical representation of a plurality of respiration rates.

A time at which RSA pacing may be relatively easier to implement and provide relatively greater therapeutic benefit is when patient 14 is sleeping or at rest. In some examples, RSA activation criteria 94 may include a target posture, e.g., lying down, and/or a threshold activity level as one or more criteria that may be satisfied if the patient is in the target posture and/or the activity level of patient 14 is below a threshold activity level. Activity level and posture of patient may be determined by processing circuitry 80 based on an accelerometer signal from a sensor 84.

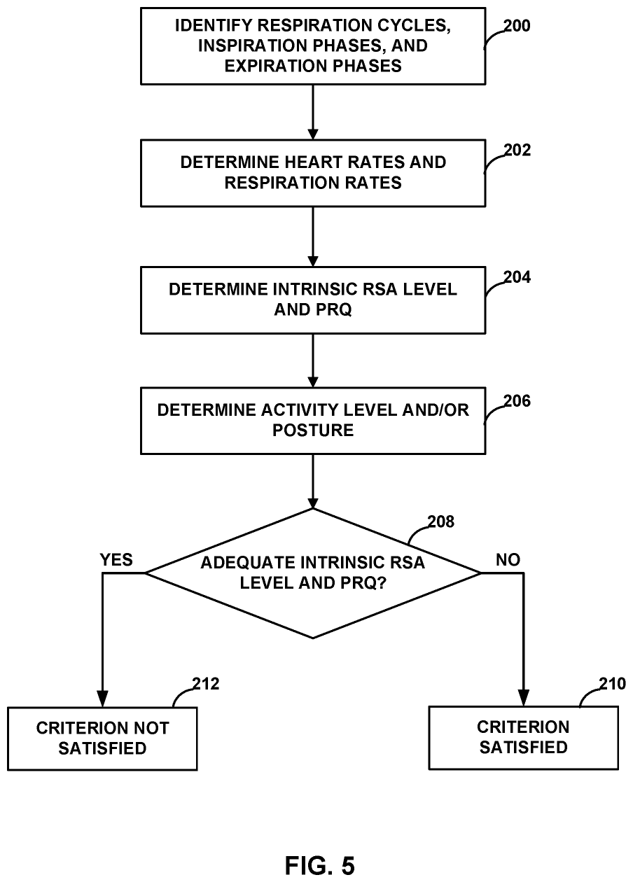

In some examples, RSA activation criteria 94 may include one or more criteria related to a degree to which patient 14 needs or would benefit from RSA pacing. For example, processing circuitry 80 may determine an intrinsic level of RSA in patient 12 based on a cardiac electrogram signal and a respiration signal of patient 12, e.g., based on heart rates during inspiration and expiration phases of one or more respiration cycles of patient 12, and a criterion may be a threshold intrinsic RSA level that is satisfied by the determined intrinsic RSA level being below the threshold. An example of determining an intrinsic RSA level is described with respect to FIG. 5.

In some examples, the one or more criteria related to a degree to which patient 14 needs or would benefit from RSA pacing are considered in conjunction with, e.g., modified based on or weighted against, other RSA activation criteria 94. For example, processing circuitry 80 may not activate RSA when intrinsic RSA is below an intrinsic RSA threshold, if respiration rate is above respiration rate threshold. As another example, processing circuitry 80 may not activate RSA when intrinsic RSA is below an intrinsic RSA threshold, if the RSA pacing rate is below a lower pacing rate threshold.

Another RSA activation criterion 94 that may relate to a degree to which patient 14 needs or would benefit from RSA pacing is a threshold level of one or more heart failure metrics or a heart failure risk score. Metrics or a risk level above a threshold may indicate worsening heart failure and a greater need for the therapeutic benefits of RSA. In some examples, processing circuitry 80 may modify one or more other RSA activation criteria 94 based on heart failure metrics or risk level being above a threshold, e.g., to favor activation of RSA pacing. For example, processing circuitry 80 may activate RSA pacing when respiration rate is not below a respiration rate threshold if heart failure metrics or risk level being above a metric or risk level threshold, because respiration rate may be due to shortness of breath associated with HF exacerbation. In some examples, processing circuitry 80 may activate RSA pacing when heart failure metrics indicate that patient is in a compensated state or a heart failure risk score is below a threshold. Since RSA pacing may be prophylactic, it would be more effective when patient is in a compensated state.

In some examples, RSA activation criteria 94 may include a threshold percentage or other amount of time of pacing, which may be satisfied if the percentage or amount during a time period, e.g., an hour or day, is less than the threshold. Overdrive RSA pacing may not be desired where pacing burden is already relatively high. The RSA activation criteria 94 described herein can be used alone or together in any suitable combination.

FIG. 5 is a flow diagram illustrating an example operation of a device to determine whether an RSA pacing mode activation criterion is satisfied. More particularly, FIG. 5 illustrates an example in which processing circuitry 80 compares an intrinsic RSA level to a threshold RSA level. Although described in the context of IMD 16, the example operation of FIG. 5 may be additionally or alternatively be performed by other devices, as described herein.

According to the example of FIG. 5, processing circuitry 80 processes a respiration signal to identify one or more respiration cycles, including the timing (e.g., start and end) of inspiration and expiration phases of the respiration cycles (200). In one example, three respiration cycles are identified and demarcated in this manner, although other numbers may be processed in other examples. Processing circuitry 80 also determines heart rates, including at least heart rates or cardiac cycle lengths during the identified respiration cycles, and one or more respiration rates or respiration cycle lengths (202).

Processing circuitry 80 determines an intrinsic RSA level and pulse respiration quotient (PRQ) based on the heart rates during the respiration cycles and the respiration rates (204). Processing circuitry 80 may determine the intrinsic RSA level based on a number of heart beats, an average heart rate, or a change in heart rates during each of the inspiration and expiration phases of the respiration cycles. In some examples, processing circuitry 80 determines intrinsic RSA level based on a comparison, e.g., difference, between a heart rate during the inspiration cycle, e.g., a maximum heart rate, and a heart rate during the expiration cycle, e.g., a minimum heart rate. Since intrinsic RSA occurs during intrinsic (non-paced) activity of the heart, processing circuitry 80 may lower a pacing rate and/or suspend pacing to allow intrinsic activity of the heart to occur to determine intrinsic RSA level.

Processing circuitry 80 may determine PRQ as a ratio of heart rate to respiratory rate, e.g., an average of heart rates divided by an average of respiration rates during the identified respiration cycles. Changes in PRQ may be related to changes in health and disease condition. Thus, PRQ may provide feedback on need for and effectiveness of therapies, such as RSA pacing. Processing circuitry 80 may also determine an activity level and/or posture of patient 14 during the identified cardiac cycles (206).

Processing circuitry 80 determines whether the intrinsic RSA level and/or PRQ are adequate (208). If processing circuitry 80 determines that the intrinsic RSA level and/or PRQ are not adequate (NO of 208), processing circuitry 80 determines that the RSA activation criterion 94 is satisfied (210). If processing circuitry 80 determines that the intrinsic RSA level and/or PRQ are adequate (YES of 208), processing circuitry 80 determines that the RSA activation criterion 94 is not satisfied (212).

Processing circuitry 80 may determine whether the intrinsic RSA level and PRQ are adequate by comparison with one or more thresholds. For example, a threshold RSA level may be an average number of heart beats, intrinsic heart rate, or heart rate change (e.g., difference between inspiration and expiration heart rates) determined across multiple respiration cycles using beats during both respiration phases. In some examples, processing circuitry 80 may adjust or otherwise determine the threshold level for intrinsic RSA based on respiratory effort, e.g., one or both of inspiration effort and expiration effort, or tidal volume. Processing circuitry 80 may adjust or otherwise determine a threshold PRQ based on the determined activity level and/or posture. In some examples, intrinsic RSA may not be known, e.g., due to cardiac pacing at a lower programmed pacing rate or sensor indicated rate that precludes an intrinsic RSA response. In such examples, processing circuitry 80 can set RSA amplitude using the intrinsic respiratory rate and a desired PRQ based on the respiration rate and lower programmed pacing rate.

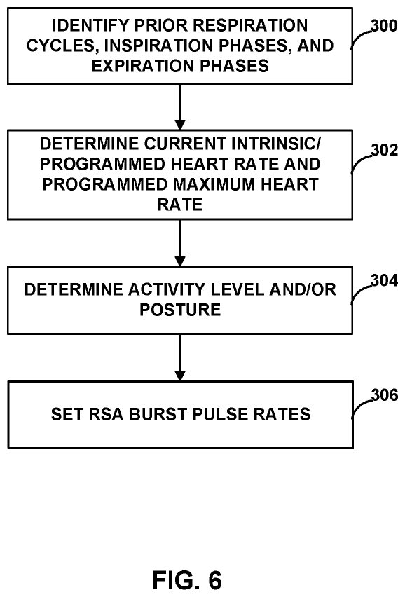

FIG. 6 is a flow diagram illustrating an example operation of a device to configure delivery of RSA pacing. Although described in the context of IMD 16, the example operation of FIG. 6 may be additionally or alternatively be performed by other devices, as described herein.

According to the example of FIG. 6, processing circuitry 80 may identify a plurality of prior respiration cycles, including timing of inspiration and expiration phases (300). Processing circuitry 80 may configure the RSA pacing burst, e.g., the starts and ends of the increasing rate ramp and decreasing rate ramp, based on the identified prior cycle timings. Processing circuitry 80 may further determine an intrinsic heart rate or programmed heart rate, e.g., a lower rate, and a programmed maximum heart rate (302). Processing circuitry 80 may configure the pacing rate ramps based on these boundaries. Processing circuitry 80 may also determine an activity level and/or posture of the patient (304). Processing circuitry 80 may configure the RSA burst pulse rates based on these determined values according to RSA pacing parameters 96 (306).

In some examples, processing circuitry 80 may configure the RSA pacing to provide an increase in instantaneous heart rate after detecting inspiration. The ramp may be sinusoidal or linear. Processing circuitry 80 may configure a rate of increase during the inspiration phase ramp based on the determined inspiration and expiration timing, programmed lower rate, and RSA amplitude. The inspiration and expiration timing may be an average of times from the past few respiration cycles. A typical inspiration phase to expiration phase ratio is 1:2.

Processing circuitry 80 may determine the number of pacing pulses during the RSA burst by averaging the difference between the RSA upper rate (programmed lower rate+the RSA programmed amplitude) and lower rate to determine how many paced beats should be in the inspiration cycle and expiration cycle, respectively. Processing circuitry 80 may configure the pacing rates during the ramp to simulate physiological sinusoidal RSA, e.g., rate ramps up during inspiration to peak RSA amplitude, and ramps back down to the programmed lower rate during expiration.

Processing circuitry 80 may modify the RSA pulse burst configuration based on PRQ, posture and/or activity. For example, processing circuitry 80 may set the lower pacing rate based on the greater of a rate associated with a desired PRQ based on sensed respiratory rate and activity/posture, or the intrinsic rate. Additionally, breathing is both voluntary and involuntary. Processing circuitry 80 may hold the RSA burst peak while patient 14 is taking deep breaths if exhalation doesn't immediately occur, e.g., within a time limit.

The disclosure includes the following non-limiting examples.

Example 1. A device comprising: therapy delivery circuitry configured to deliver cardiac pacing pulses to a heart of a patient via a plurality of electrodes; sensing circuitry configured to sense one or more parameters of the patient; and processing circuitry configured to: determine that one or more criteria for activation of a respiratory sinus arrhythmia (RSA) mode are satisfied based on the one or more parameters of the patient; and control the therapy delivery circuitry to deliver the cardiac pacing pulses according to the RSA mode based on the determination, wherein delivery of the cardiac pacing pulses according to the RSA mode includes increasing a rate of the cardiac pacing pulses during an inspiration phase of a respiration cycle of the patient, and decreasing a rate of the cardiac pacing pulses during an expiration phase of the respiration cycle of the patient.

Example 2. The device of example 1, wherein the processing circuitry is configured to control the therapy delivery circuitry deliver the cardiac pacing pulses according to one or more of a demand pacing mode, a rate responsive pacing mode, or a cardiac resynchronization therapy (CRT) pacing mode.

Example 3. The device of example 1 or 2, wherein the sensing circuitry is configured to sense a cardiac electrogram signal of the patient via the plurality of electrodes, and the processing circuitry is configured to determine an intrinsic heart rate of the patient based on the cardiac electrogram signal, and wherein, to determine that the one or more criteria for activation of the RSA mode are satisfied, the processing circuitry is configured to determine that the intrinsic heart rate is below a threshold heart rate.

Example 4. The device of any one or more of examples 1 to 3, wherein the sensing circuitry is configured to receive a signal from an accelerometer, and the processing circuitry is configured to determine at least one of a posture of the patient or an activity level of the patient based on the signal from the accelerometer, and wherein, to determine that the one or more criteria for activation of the RSA mode are satisfied, the processing circuitry is configured to at least one of: determine that the posture of the patient is lying down; or determine that the activity level of the patient is below a threshold activity level.

Example 5. The device of any one or more of examples 1 to 4, wherein the sensing circuitry is configured to sense a respiration signal of the patient, and the processing circuitry is configured to determine a respiration rate of the patient based on the respiration signal, and wherein, to determine that the one or more criteria for activation of the RSA mode are satisfied, the processing circuitry is configured to determine that the respiration rate is below a threshold respiration rate.

Example 6. The device of example 1 or 2, wherein the sensing circuitry is configured to sense a cardiac electrogram signal via the plurality electrodes and a respiration signal, and the processing circuitry is configured to: identify one or more prior respiration cycles based on the respiration signal; and for each prior respiration cycle of the one or more prior respiration cycles: identify an inspiration phase and an expiration phase; and determine heart rates during the inspiration phase and heart rates during the expiration phase based on the cardiac electrogram signal, and wherein, to determine that the one or more criteria for activation of the RSA mode are satisfied, the processing circuitry is configured to: determine an intrinsic level of RSA based on the heart rates during the inspiration phase and the heart rates during the expiration phase for the one or more prior respiration cycles; and determine that the intrinsic level of RSA is below a threshold level of RSA.

Example 7. The device of example 6, wherein the processing circuitry is configured to: determine at least one of an inspiration effort, an expiration effort, or a tidal volume based on the respiration signal during the one or more prior respiration cycles; and determine the threshold level of RSA based on the at least one of the expiratory effort or the tidal volume.

Example 8. The device of example 6 or 7, wherein the sensing circuitry is configured to receive a signal from an accelerometer, and the processing circuitry is configured to: determine at least one of a posture of the patient or an activity level of the patient based on the signal from the accelerometer; and determine the threshold level of RSA based on the at least one of the posture or the activity level.

Example 9. The device of any one or more of examples 6 to 8, wherein the processing circuitry is configured to: determine respiration rates based on the respiration signal; determine a quotient based on the heart rates and the respiration rates; and determine whether the one or more criteria for activation of the RSA mode are satisfied based on the quotient.

Example 10. The device of example 9, wherein the sensing circuitry is configured to receive a signal from an accelerometer, and the processing circuitry is configured to: determine at least one of a posture of the patient or an activity level of the patient based on the signal from the accelerometer; determine a threshold quotient based on the at least one of the posture or the activity level; and determine whether the one or more criteria for activation of the RSA mode are satisfied based on a comparison of the quotient to the threshold quotient.

Example 11. The device of any one or more of examples 1 to 10, wherein the processing circuitry is configured to: determine one or more heart failure metrics based on the one or more parameters; and determine that the one or more criteria for activation of the RSA mode are satisfied based on the one or more heart failure metrics.

Example 12. The device of example 11, wherein, to determine that the one or more criteria for activation of the RSA mode are satisfied based on the one or more heart failure metrics, the processing circuitry is configured to: determine a heart failure risk score based on the one or more heart failure metrics; and determine that the heart failure risk score exceeds a threshold risk score.

Example 13. The device of any one or more of example 1 to 12, wherein, to determine that the one or more criteria for activation of the RSA mode are satisfied, the processing circuitry is configured to determine that an amount of cardiac pacing delivered to the patient is less than a threshold amount of pacing.

Example 14. The device of example 1, wherein the sensing circuitry is configured to sense a cardiac electrogram signal of the patient via the plurality of electrodes, and the processing circuitry is configured to: determine an intrinsic heart rate of the patient based on the cardiac electrogram signal; and set the rates of the cardiac pacing pulses delivered according to the RSA mode based on the intrinsic heart rate of the patient.

Example 15. The device of example 14, wherein the processing circuitry is configured to set the rates of the cardiac pacing pulses delivered according to the RSA mode based on a predetermined maximum rate of cardiac pacing pulses for the RSA mode.

Example 16. The device of example 14 or 15, wherein the sensing circuitry is configured to sense a respiration signal of the patient, and the processing circuitry is configured to: determine at least one of an inspiration effort, an expiration effort, or a tidal volume based on the respiration signal; and set the rates of the cardiac pacing pulses delivered according to the RSA mode based on the at least one of the inspiration effort, the expiration effort, or the tidal volume.

Example 17. The device of any one or more of example 1 to 16, further comprising a housing for the therapy delivery circuitry, the sensing circuitry, and the processing circuitry, wherein the housing is configured for implantation within the patient.

Example 18. A method comprising: sensing one or more parameters of a patient; determining that one or more criteria for activation of a respiratory sinus arrhythmia (RSA) mode are satisfied based on the one or more parameters of the patient; and delivering cardiac pacing pulses according to the RSA mode based on the determination, wherein delivery of the cardiac pacing pulses according to the RSA mode includes increasing a rate of the cardiac pacing pulses during an inspiration phase of a respiration cycle of the patient, and decreasing a rate of the cardiac pacing pulses during an expiration phase of the respiration cycle of the patient.

Example 19. The method of example 18, further comprising delivering the cardiac pacing pulses according to one or more of a demand pacing mode, a rate responsive pacing mode, or a cardiac resynchronization therapy (CRT) pacing mode.

Example 20. The method of example 18 or 19, wherein sensing the one or more parameters of the patient comprises sensing a cardiac electrogram signal of the patient, the method further comprising determining an intrinsic heart rate of the patient based on the cardiac electrogram signal, and wherein determining that the one or more criteria for activation of the RSA mode are satisfied comprises determining that the intrinsic heart rate is below a threshold heart rate.

Example 21. The method of any one or more of examples 18 to 20, wherein sensing the one or more parameters of the patient comprises: receiving a signal from an accelerometer; and determining at least on one of a posture of the patient or an activity level of the patient based on the signal from the accelerometer, and wherein determining that the one or more criteria for activation of the RSA mode are satisfied comprises at least one of: determining that the posture of the patient is lying down; or determining that the activity level of the patient is below a threshold activity level.

Example 22. The method of any one or more of examples 18 to 21, wherein sensing the one or more parameters of the patient comprises: sensing a respiration signal of the patient; and determining a respiration rate of the patient based on the respiration signal, wherein determining that the one or more criteria for activation of the RSA mode are satisfied comprises determining that the respiration rate is below a threshold respiration rate.

Example 23. The method of example 18 or 22, wherein sensing the one or more parameters of the patient comprises sensing a cardiac electrogram signal and a respiration signal, the method further comprising: identifying one or more prior respiration cycles based on the respiration signal; and for each prior respiration cycle of the one or more prior respiration cycles: identifying an inspiration phase and an expiration phase; and determining heart rates during the inspiration phase and heart rates during the expiration phase based on the cardiac electrogram signal, and wherein determining that the one or more criteria for activation of the RSA mode are satisfied comprises: determining an intrinsic level of RSA based on the heart rates during the inspiration phase and the heart rates during the expiration phase for the one or more prior respiration cycles; and determining that the intrinsic level of RSA is below a threshold level of RSA.

Example 24. The method of example 23, further comprising: determining at least one of an inspiration effort, an expiration effort, or a tidal volume based on the respiration signal during the one or more prior respiration cycles; and determining the threshold level of RSA based on the at least one of the expiratory effort or the tidal volume.

Example 25. The method of example 23 or 24, further comprising: determining at least one of a posture of the patient or an activity level of the patient based on a signal from an accelerometer; and determining the threshold level of RSA based on the at least one of the posture or the activity level.

Example 26. The method of any one or more of examples 23 to 25, further comprising: determining respiration rates based on the respiration signal; determining a quotient based on the heart rates and the respiration rates; and determining whether the one or more criteria for activation of the RSA mode are satisfied based on the quotient.

Example 27. The method of example 26, further comprising: determining at least one of a posture of the patient or an activity level of the patient based on a signal from an accelerometer; determining a threshold quotient based on the at least one of the posture or the activity level; and determining whether the one or more criteria for activation of the RSA mode are satisfied based on a comparison of the quotient to the threshold quotient.

Example 28. The method of any one or more of examples 18 to 27, further comprising: determining one or more heart failure metrics based on the one or more parameters; and determining that the one or more criteria for activation of the RSA mode are satisfied based on the one or more heart failure metrics.

Example 29. The method of example 28, wherein determining that the one or more criteria for activation of the RSA mode are satisfied based on the one or more heart failure metrics comprises: determining a heart failure risk score based on the one or more heart failure metrics; and determining that the heart failure risk score exceeds a threshold risk score.

Example 30. The method of any one or more of examples 18 to 29, wherein determining that the one or more criteria for activation of the RSA mode are satisfied comprises determining that an amount of cardiac pacing delivered to the patient is less than a threshold amount of pacing.

Example 31. The method of example 18, further comprising: sensing a cardiac electrogram signal of the patient via the plurality of electrodes; determining an intrinsic heart rate of the patient based on the cardiac electrogram signal; and setting the rates of the cardiac pacing pulses delivered according to the RSA mode based on the intrinsic heart rate of the patient.

Example 32. The method of example 31, further comprising setting the rates of the cardiac pacing pulses delivered according to the RSA mode based on a predetermined maximum rate of cardiac pacing pulses for the RSA mode.

Example 33. The method of example 31 or 32, further comprising: sensing a respiration signal of the patient; determining at least one of an inspiration effort, an expiration effort, or a tidal volume based on the respiration signal; and setting the rates of the cardiac pacing pulses delivered according to the RSA mode based on the at least one of the inspiration effort, the expiration effort, or the tidal volume.

Example 34. The method of any one or more of examples 18 to 33, wherein an implantable medical device implanted within the patient senses the one or more parameters of a patient, determines that the one or more criteria for activation of the RSA mode are satisfied, and delivers cardiac pacing pulses according to the RSA mode based on the determination.

Example 35. A non-transitory computer-readable storage medium comprising program instructions that, when executed by processing circuitry of a device, cause the device to perform the method of any one or more of examples 18 to 34.

Various examples have been described. These and other examples are within the scope of the following claims.

1. A device comprising:
therapy delivery circuitry configured to deliver cardiac pacing pulses to a heart of a patient via a plurality of electrodes;
sensing circuitry configured to sense one or more parameters of the patient; and
processing circuitry configured to:
determine that one or more criteria for activation of a respiratory sinus arrhythmia (RSA) mode are satisfied based on the one or more parameters of the patient; and
control the therapy delivery circuitry to deliver the cardiac pacing pulses according to the RSA mode based on the determination, wherein delivery of the cardiac pacing pulses according to the RSA mode includes increasing a rate of the cardiac pacing pulses during an inspiration phase of a respiration cycle of the patient, and decreasing a rate of the cardiac pacing pulses during an expiration phase of the respiration cycle of the patient.

2. The device of claim 1, wherein the processing circuitry is configured to control the therapy delivery circuitry to deliver the cardiac pacing pulses according to one or more of a demand pacing mode, a rate responsive pacing mode, or a cardiac resynchronization therapy (CRT) pacing mode.

3. The device of claim 1, wherein the sensing circuitry is configured to sense a cardiac electrogram signal of the patient via the plurality of electrodes, and the processing circuitry is configured to determine an intrinsic heart rate of the patient based on the cardiac electrogram signal, and wherein, to determine that the one or more criteria for activation of the RSA mode are satisfied, the processing circuitry is configured to determine that the intrinsic heart rate is below a threshold heart rate.

4. The device of claim 1, wherein the sensing circuitry is configured to receive a signal from an accelerometer, and the processing circuitry is configured to determine at least one of a posture of the patient or an activity level of the patient based on the signal from the accelerometer, and wherein, to determine that the one or more criteria for activation of the RSA mode are satisfied, the processing circuitry is configured to at least one of:
determine that the posture of the patient is lying down; or
determine that the activity level of the patient is below a threshold activity level.

5. The device of claim 1, wherein the sensing circuitry is configured to sense a respiration signal of the patient, and the processing circuitry is configured to determine a respiration rate of the patient based on the respiration signal, and wherein, to determine that the one or more criteria for activation of the RSA mode are satisfied, the processing circuitry is configured to determine that the respiration rate is below a threshold respiration rate.

6. The device of claim 1, wherein the sensing circuitry is configured to sense a cardiac electrogram signal via the plurality electrodes and a respiration signal, and the processing circuitry is configured to:
identify one or more prior respiration cycles based on the respiration signal; and
for each prior respiration cycle of the one or more prior respiration cycles:
identify an inspiration phase and an expiration phase; and
determine heart rates during the inspiration phase and heart rates during the expiration phase based on the cardiac electrogram signal, and
wherein, to determine that the one or more criteria for activation of the RSA mode are satisfied, the processing circuitry is configured to:
determine an intrinsic level of RSA based on the heart rates during the inspiration phase and the heart rates during the expiration phase for the one or more prior respiration cycles; and
determine that the intrinsic level of RSA is below a threshold level of RSA.

7. The device of claim 6, wherein the processing circuitry is configured to:
determine at least one of an inspiration effort, an expiration effort, or a tidal volume based on the respiration signal during the one or more prior respiration cycles; and
determine the threshold level of RSA based on the at least one of the expiratory effort or the tidal volume.

8. The device of claim 6, wherein the sensing circuitry is configured to receive a signal from an accelerometer, and the processing circuitry is configured to:
determine at least one of a posture of the patient or an activity level of the patient based on the signal from the accelerometer; and
determine the threshold level of RSA based on the at least one of the posture or the activity level.

9. The device of claim 6, wherein the processing circuitry is configured to:
determine respiration rates based on the respiration signal;
determine a quotient based on the heart rates and the respiration rates; and
determine whether the one or more criteria for activation of the RSA mode are satisfied based on the quotient.

10. The device of claim 9, wherein the sensing circuitry is configured to receive a signal from an accelerometer, and the processing circuitry is configured to:
determine at least one of a posture of the patient or an activity level of the patient based on the signal from the accelerometer;
determine a threshold quotient based on the at least one of the posture or the activity level; and
determine whether the one or more criteria for activation of the RSA mode are satisfied based on a comparison of the quotient to the threshold quotient.

11. The device of claim 1, wherein the processing circuitry is configured to:
determine one or more heart failure metrics based on the one or more parameters; and
determine that the one or more criteria for activation of the RSA mode are satisfied based on the one or more heart failure metrics.

12.-13. (canceled)

14. The device of claim 1, wherein the sensing circuitry is configured to sense a cardiac electrogram signal of the patient via the plurality of electrodes, and the processing circuitry is configured to:
determine an intrinsic heart rate of the patient based on the cardiac electrogram signal; and
set the rates of the cardiac pacing pulses delivered according to the RSA mode based on the intrinsic heart rate of the patient.

15. (canceled)

16. The device of claim 14, wherein the sensing circuitry is configured to sense a respiration signal of the patient, and the processing circuitry is configured to:
determine at least one of an inspiration effort, an expiration effort, or a tidal volume based on the respiration signal; and
set the rates of the cardiac pacing pulses delivered according to the RSA mode based on the at least one of the inspiration effort, the expiration effort, or the tidal volume.

17. The device of claim 1, further comprising a housing for the therapy delivery circuitry, the sensing circuitry, and the processing circuitry, wherein the housing is configured for implantation within the patient.

18. A method comprising:
sensing one or more parameters of a patient;
determining that one or more criteria for activation of a respiratory sinus arrhythmia (RSA) mode are satisfied based on the one or more parameters of the patient; and
delivering cardiac pacing pulses according to the RSA mode based on the determination, wherein delivery of the cardiac pacing pulses according to the RSA mode includes increasing a rate of the cardiac pacing pulses during an inspiration phase of a respiration cycle of the patient, and decreasing a rate of the cardiac pacing pulses during an expiration phase of the respiration cycle of the patient.

19. The method of claim 18, further comprising delivering the cardiac pacing pulses according to one or more of a demand pacing mode, a rate responsive pacing mode, or a cardiac resynchronization therapy (CRT) pacing mode.

20. The method of claim 18, wherein sensing the one or more parameters of the patient comprises sensing a cardiac electrogram signal of the patient, the method further comprising determining an intrinsic heart rate of the patient based on the cardiac electrogram signal, and wherein determining that the one or more criteria for activation of the RSA mode are satisfied comprises determining that the intrinsic heart rate is below a threshold heart rate.

21. The method of claim 18, wherein sensing the one or more parameters of the patient comprises:
receiving a signal from an accelerometer; and
determining at least on one of a posture of the patient or an activity level of the patient based on the signal from the accelerometer, wherein determining that the one or more criteria for activation of the RSA mode are satisfied comprises at least one of:
determining that the posture of the patient is lying down; or
determining that the activity level of the patient is below a threshold activity level.

22. The method of claim 18, wherein sensing the one or more parameters of the patient comprises:
sensing a respiration signal of the patient; and
determining a respiration rate of the patient based on the respiration signal, wherein determining that the one or more criteria for activation of the RSA mode are satisfied comprises determining that the respiration rate is below a threshold respiration rate.

23. A non-transitory computer-readable storage medium comprising program instructions that, when executed by processing circuitry of a device, cause the processing circuitry to:
control sensing circuitry of the device to sense one or more parameters of a patient;
determine that one or more criteria for activation of a respiratory sinus arrhythmia (RSA) mode are satisfied based on the one or more parameters of the patient; and
control therapy delivery circuitry of the device to deliver the cardiac pacing pulses according to the RSA mode based on the determination, wherein delivery of the cardiac pacing pulses according to the RSA mode includes increasing a rate of the cardiac pacing pulses during an inspiration phase of a respiration cycle of the patient, and decreasing a rate of the cardiac pacing pulses during an expiration phase of the respiration cycle of the patient.
